(19) United States
(12) Patent Application Publication
(10) Pub. No.: US 2019/0255508 A1
(43) Pub. Date: Aug. 22, 2019

(54) PATCH AND MANUFACTURING METHOD THEREOF

(71) Applicant: AlphaBio Centrix LLC, Henderson, NV (US)

(72) Inventor: Richard EATON, Henderson, NV (US)

(73) Assignee: AlphaBio Centrix LLC, Henderson, NV (US)

(21) Appl. No.: 16/367,271

(22) Filed: Mar. 28, 2019

Related U.S. Application Data
(63) Continuation-in-part of application No. 15/595,950, filed on May 16, 2017.

(51) Int. Cl. B01J 19/12; H02S 99/00; H01L 31/048; H01L 31/18

(52) U.S. Cl. CPC B01J 19/12; H02S 99/00; B01J 2219/0877; H01L 31/18; B01J 2219/0803; H01L 31/048

(57) ABSTRACT

Disclosed in the present invention is a patch for emitting electromagnetic energy. The patch comprises: a first layer made of a material that is capable of storing and emitting electromagnetic energy, a second layer disposed above the first layer and made of a material that is capable of storing and emitting electromagnetic energy, an energy harvesting layer disposed above the second layer, a transparent dome may be disposed above the solar panel, and an adhesive layer disposed below the first layer. A method for manufacturing the patch is also disclosed.

### TECHNICAL FIELD

The present invention relates to the field of storing and releasing electromagnetic energy in radio frequency ranges, more particularly, to a patch that can store electromagnetic energy and release the electromagnetic energy for different applications.

### BACKGROUND

Electromagnetic energy is used in many fields. In certain applications energy in electromagnetic waves is converted to a different form. For example, electromagnetic waves can be used to heat an element or a composition. Electromagnetic energy may also be used to cause changes in a composition and change in composition's elements.

Automobile smog or emission test failures may happen due to the impurities in petroleum that release toxic elements and carbon deposits into the environment when combusted. These toxic elements are known as hydrocarbons (HC), nitrogen oxides (NOₓ), carbon dioxide (CO₂), and carbon monoxide (CO), and are measured in ppm (parts-per-million).

Unburned hydrocarbons and nitrogen oxides react in the atmosphere to form photochemical smog. Smog is highly oxidizing in the environment and the prime cause of eye and throat irritation, bad odor, plant damage, and decreased visibility. Carbon Monoxide impairs blood capability to carry oxygen to the brain, resulting in slower reaction times and impaired judgment. Carbon Dioxide results in increasing the greenhouse effect and warming the planet.

These problems led to the development of the Catalytic Converter, which is an emissions control device that converts toxic gasses and pollutants in the exhaust gas to less toxic pollutants by catalyzing a redox reaction (an oxidation and a reduction reaction). Catalytic converters are used with internal combustion engines fueled by either petrol (gasoline) or diesel-including lean-burn engines. The first widespread introduction of catalytic converters was in the United States automobile market.

Although catalytic converters are most commonly applied to exhaust systems in automobiles, this is usually in response to government regulation, either through direct environmental regulation or through health and safety regulations.

### SUMMARY OF THE INVENTION

The present invention relates to a patch developed through the applied science of quantum mechanics to store electromagnetic energy and release it slowly for different applications.

According to one embodiment of the invention, a patch is constructed by using material that is capable of storing electromagnetic energy. The patch is constructed by adding various materials.

According to another embodiment of the invention, electromagnetic signals are applied to the patch at different frequencies such that the electromagnetic energy is stored in the patch.

According to another embodiment of the invention, a patch is constructed using electromagnetic energy storing material and energy harvesting material is used to keep the stored electromagnetic energy last longer than it would without the energy harvesting material.

According to another embodiment of the invention, the energy harvesting material can harvest electromagnetic energy from the environment or ambient surroundings and releases that energy to the material that is capable of storing electromagnetic energy to prolong the time that electromagnetic energy is released from the patch.

According to another embodiment, the energy harvesting material can harvest energy from light and releases that energy to the electromagnetic energy storing material to prolong the time that electromagnetic energy is released from the patch.

According to another embodiment, the patch is placed on a human body and releases the stored electromagnetic energy to a human body.

According to another embodiment, the patch is placed on a fuel tank to increase fuel efficiency and reduce carbon-dioxide emissions.

According to one aspect of the present invention, a patch for emitting electromagnetic energy is provided. The patch comprises: a first layer made of a first material that is capable of storing electromagnetic energy, and a second layer disposed above the first layer and made of a second material that is capable of storing electromagnetic energy.

The patch may further comprise a third layer made of a third material that is capable of harvesting electromagnetic energy, wherein the energy harvesting material is disposed above the second layer.

The patch may further comprise an adhesive layer that is disposed below the first layer.

The first layer may be made of crystalline carbon, and the second layer may be made of crystalline carbon.

The third layer may be a solar panel for harvesting electromagnetic energy in light.

The patch may further comprise a transparent dome disposed above the solar panel.

The patch may further comprise an adhesive layer disposed below the first layer.

The first layer and the second layer may be treated by a series of electromagnetic waves at predetermined frequencies.

The predetermined frequencies may include at least one selected from the group consisting of 2720 Hz, 2170 Hz, 1800 Hz, 465 Hz, 644 Hz, 660 Hz, and 19180 Hz.

The thickness of the first layer may be about 0.5 mil.

The thickness of the second layer may be about 0.5 mil.

The thickness of the solar panel may be about 1 mil.

The thickness of the transparent dome may be about 1/16 inch.

The crystalline carbon material is a radio frequency sensitive material.

The crystalline carbon material may be blended with PET, wherein the PET is produced by polymerization of ethylene glycol and terephthalic acid.

According to another aspect of the present invention, a method for manufacturing a patch is provided. The method comprises: forming a first layer that is capable of storing electromagnetic energy; forming, above the first layer, a second layer that is capable of storing electromagnetic energy; and applying a series of electromagnetic waves at predetermined frequencies to the first and second layers.

The electromagnetic waves at predetermined frequencies may be applied under a vacuum condition or approximate vacuum condition.

The method may further comprise forming a third layer that is capable of harvesting electromagnetic energy in the light above the second layer.

The third layer may be a solar panel.

The method may further comprise forming a transparent dome above the third layer.

The method may further comprise forming an adhesive layer below the first layer.

The first layer may be made of a blend of a crystalline carbon material and PET, wherein the PET is produced by polymerization of ethylene glycol and terephthalic acid.

The second layer may be made of a blend of a crystalline carbon material and PET, wherein the PET is produced by polymerization of ethylene glycol and terephthalic acid.

The step of applying a series of electromagnetic waves at predetermined frequencies to the first and second layers under a vacuum condition may comprise: applying an electromagnetic wave at a frequency of 2720 Hz to the first and second layers for about 2-3 seconds; applying an electromagnetic wave at a frequency of 2170 Hz to the first and second layers for about 2-3 seconds; applying an electromagnetic wave at a frequency of 1800 Hz to the first and second layers for about 2-3 seconds; applying an electromagnetic wave at a frequency of 465 Hz to the first and second layers for about 2-3 seconds; applying an electromagnetic wave at a frequency of 644 Hz to the first and second layers for about 2-3 seconds; applying an electromagnetic wave at a frequency of 660 Hz to the first and second layers for about 2-3 seconds; and applying an electromagnetic wave at a frequency of 19180 Hz to the first and second layers for about 2-3 seconds.

The series of electromagnetic waves at predetermined frequencies may be applied sequentially without time interval or a time interval can be introduced between applying electromagnetic waves.

The crystalline carbon material may be radio frequency sensitive.

### BRIEF DESCRIPTION OF DRAWINGS

The above and other objects, features and other advantages of the present invention will be more clearly understood from the following detailed description taken in conjunction with the accompanying drawings, in which:

FIG. 1 is a cross-sectional view of a patch in accordance with a first embodiment of the present application.

FIG. 2 is a cross-sectional view of a patch in accordance with a second embodiment of the present application.

FIG. 3 is a cross-sectional view of a patch in accordance with a third embodiment of the present application.

FIG. 4 is a cross-sectional view of a patch in accordance with a fourth embodiment of the present application.

FIG. 5 is a cross-sectional view of a patch in accordance with a fifth embodiment of the present application.

FIG. 6 is a cross-sectional view of a patch in accordance with a sixth embodiment of the present application.

FIG. 7 is a top view of the patch in accordance with the second embodiment of the present application.

FIG. 8 is a structural schematic diagram of equipment for manufacturing the patch in accordance with the present application.

FIG. 9 is a flow chart of a method for manufacturing the patch for fuel treatment in accordance with a seventh embodiment of the present application.

FIG. 10 is a flow chart of a process of applying electromagnetic waves in accordance with the seventh embodiment of the present application.

FIG. 11 is a flow chart of a method for manufacturing the patch for fuel treatment in accordance with an eighth embodiment of the present application.

FIG. 12 depicts the soot volume fraction distributions in the flame at HAB (Heights Above the Burner) 15 cm for the baseline and two “chip” (or patch) test campaigns, represented as baseline, CHIP Test 1, CHIP Test 2, respectively.

### DETAILED DESCRIPTION OF EMBODIMENTS

Hereinafter, a patch in accordance with exemplary embodiments of the present application will be described with reference to the accompanying drawings. During the process, a thickness of lines, a size of components, or the like, illustrated in the drawings may be exaggeratedly illustrated for clearness and convenience of explanation.

Further, the following terminologies are defined in consideration of the functions in the present invention and may be construed in different ways by intention or practice of users and operators. Therefore, the definitions of terms used in the present description should be construed based on the contents throughout the specification.

In addition, the following embodiments are not limited to the scope of the present invention but illustrated only the components included in the claims of the present invention. It will be appreciated that embodiments including components which are included in the spirit of the specification of the present invention and may be substituted into equivalents in the components of the claims may be included in the scope of the present invention.

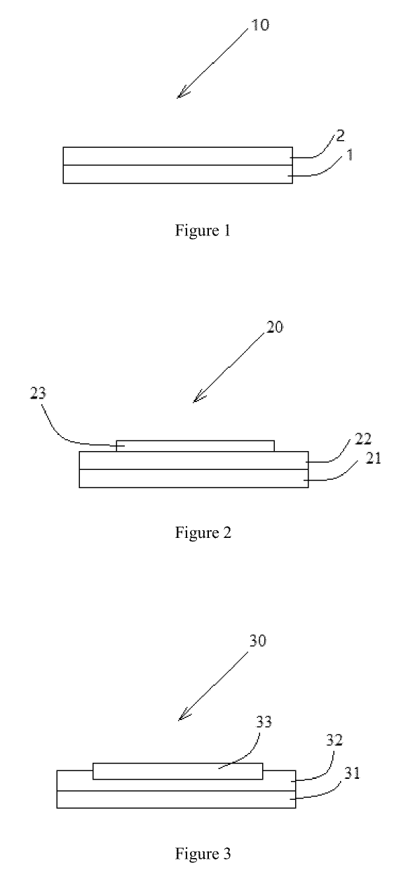

FIG. 1 shows patch 10 in accordance with the first embodiment, in which patch 10 includes first layer 1 and second layer 2 disposed above first layer 1. First layer 1 is made of a first material that is capable of storing electromagnetic energy and slowly releasing the stored electromagnetic energy. Second layer 2 is made of a material that is capable of storing electromagnetic energy and slowly releasing the stored electromagnetic energy.

In this embodiment, both the first and second materials are crystalline carbon. The crystalline carbon material indicates a carbon allotrope and can include at least one of graphite, graphene, and Fullerene.

In another embodiment, the crystalline carbon material can include fullerene. Fullerene is an allotrope of carbon in the form of a hollow sphere, ellipsoid, tube, and many other shapes and sizes. Spherical fullerenes, also referred to as Buckminsterfullerenes or Buckyballs, resemble the balls used in soccer. Cylindrical fullerenes are also called carbon nanotubes (buckytubes). Fullerenes are similar in structure to graphite, which is composed of stacked graphene sheets of linked hexagonal rings.

Examples of fullerene can include Buckyball clusters (smallest member is C₂₀ (unsaturated version of dodecahedrane) and the most common is C₆₀), Nanotubes (hollow tubes of very small dimensions, having single or multiple walls), Megatubes (larger in diameter than nanotubes and prepared with walls of different thickness), fullerene polymers (chain, two-dimensional and three-dimensional polymers formed under high-pressure high-temperature conditions), nano onions (spherical particles based on multiple carbon layers surrounding a buckyball core), linked ball-and-chain dimers (two buckyballs linked by a carbon chain), and others.

The first and second materials can be a blend of the crystalline carbon material and PET (polyethylene terephthalate), wherein the PET is produced by polymerization of ethylene glycol and terephthalic acid. PET can provide the improved distribution of the crystalline carbon material in the first and second materials, and protect the crystalline carbon material and thus improve the durability of the first and second materials. When ethylene glycol and terephthalic acid are heated together under the influence of chemical catalysts, ethylene glycol and terephthalic acid produce PET in the form of a molten, viscous mass that can be spun directly into fibers or solidified for later processing as a variety of shapes.

Before forming first and second layers 1, 2, the crystalline carbon material is treated by a series of electromagnetic waves at predetermined frequencies, including but not limited to, 2720 Hz, 2170 Hz, 1800 Hz, 465 Hz, 644 Hz, 660 Hz, and 19180 Hz (details of this treatment will be further described hereinafter). This specific material has sufficient carbon crystalline elements so that it will hold an energetic flux (i.e., electromagnetic flux), which is the rate of transfer of energy through a surface, because of its conductive properties. The quantity of the energetic flux depends on the sub-harmonic signals. This material has a carbon base and has the capability of retention like silicone, and thus is the preferred solution component for conductive use as energetic flux.

In some embodiments, the crystalline carbon material is radio frequency sensitive.

In some embodiments, first layer 1 and second layer 2 are formed as a whole.

In some embodiments, first layer 1 is made using a material which is different than the material used for second layer 2.

FIG. 2 shows patch 20 in accordance with the second embodiment. In this embodiment, patch 20 comprises first layer 21, second layer 22 disposed above first layer 21, and third layer 23 disposed above second layer 22. As first layer 21 and second layer 22 are similar to first layer 1 and second layer 2 of the first embodiment that has been described with reference to FIG. 1, a description thereof will be omitted. Third layer 23 is used to supplement the electromagnetic energy stored in first and second layers 21, 22 so as to prolong the time that electromagnetic energy released from patch 20. Third layer 23 material is made of a third material that is capable of harvesting electromagnetic energy from the external environment or ambient surroundings (such as light) and releasing that electromagnetic energy to first layer 21 and second layer 22 that hold or store electromagnetic energy.

In this embodiment, third layer 23 is a solar panel, which is configured for stabilizing the duration of time of patch 20 by separating a spectrum of light called the “visible electromagnetic spectrum” to provide an additional power to further the effect of the electromagnetic energy. This solar assisted technology works with even minimal daylight and has an expected lifespan of over one year. In some embodiment, solar panel 3 is flexible, lightweight, and about 0.5 inch in diameter.

In some embodiments, third layer 23 is made of a material that harvests energy (such as solar energy, magnetic energy, electrical energy, wind energy, water energy and so on) from the external environment and converts the external energy into electromagnetic energy that can be used by first layer 21 and second layer 22.

In some embodiments, there are two or more third layers 23.

In some embodiments, the material used in third layer 23 is similar to the material used in first layer 21 or second layer 22.

FIG. 3 shows patch 30 in accordance with the third embodiment of the present application, in which third layer 33 is embedded in second layer 32.

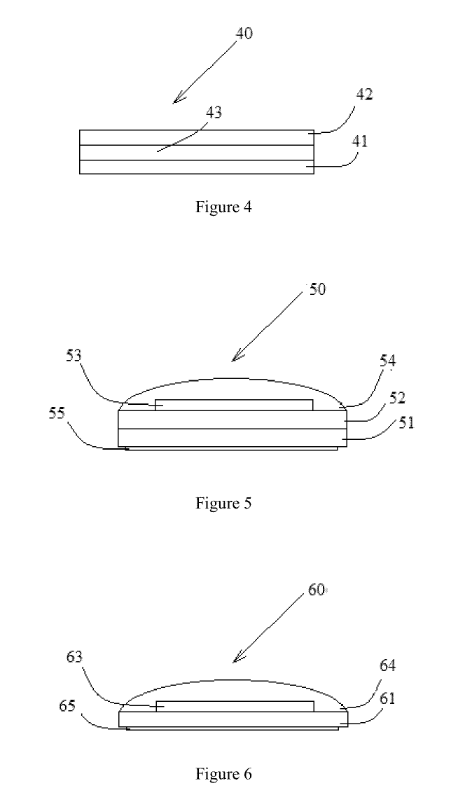

FIG. 4 shows patch 40 in accordance with the fourth embodiment of the present application, in which third layer 43 is sandwiched by first layer 41 and second layer 42.

FIG. 5 shows patch 50 in accordance with the fifth embodiment of the present application, in which patch 50 further comprises transparent dome 54 disposed above third layer 53 and adhesive layer 55 disposed below first layer 51. As first layer 51, second layer 52, third layer 53 are similar to first layer 1, second layer 2 according to the first embodiment, and third layer 23 according to the second embodiment, a description thereof will be omitted.

Transparent dome 54 is used to provide extra protection for third layer 53. In some embodiments, transparent dome 54 is made of transparent plastic.

Adhesive layer 55 is used to adhere patch 50 to the target. Adhesive layer 55 can be made of any adhesive materials.

In some embodiments, the thickness of the first layer is about 0.5 mil. In some embodiments, the thickness of the second layer is about 0.5 mil. In some embodiments, the thickness of the third layer is about 1 mil. In some embodiments, the thickness of the transparent dome is about 1/16 inch.

FIG. 6 shows patch 60 in accordance with the sixth embodiment of the present application. Compared with the fifth embodiment, patch 60 according to the sixth embodiment only comprises one layer, i.e., first layer 61 that is made of a material (such as crystalline carbon material) that is capable of storing electromagnetic energy and slowly releasing the stored electromagnetic energy. Patch 60 further comprises an additional layer 63 disposed above first layer 61, transparent dome 64 disposed above additional layer 63, and adhesive layer 65 disposed below first layer 61. As first layer 61, additional layer 63, transparent dome 64, and adhesive layer 65 are similar to the first layer, third layer, transparent dome, and adhesive layer according to previous embodiments, a description thereof will be omitted

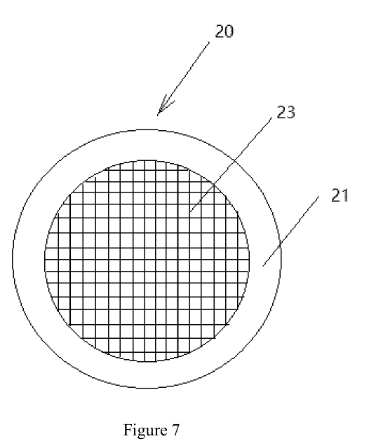

FIG. 7 is a top view of patch 20 in accordance with the second embodiment of the present application.

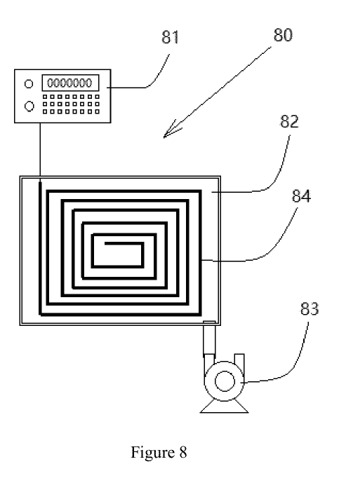

FIG. 8 is a structural schematic diagram of equipment for manufacturing the solar assisted environmental fuel patch in accordance with the present application. Referring to FIG. 7, equipment 80 includes electromagnetic wave generator 81, container 82, vacuum pump 83, and coil 84.

Electromagnetic wave generator 81 is configured to generate electromagnetic waves with various wavelengths or frequencies. Container 82 is a sealed box in which the first and second layers are subject to the electromagnetic wave treatment. Vacuum pump 83 is used to create vacuum or partial vacuum in container 82 such that the electromagnetic waves can be applied to the first and second layers under a vacuum condition. Coil 84 is provided at the bottom of container 82 to transmit the electromagnetic waves generated by electromagnetic wave generator 81. The output of electromagnetic wave generator 81 is connected to coil 84. The interior of container 82 is communicated with vacuum pump 83 via a suction pipe. In some embodiments, electromagnetic wave generator 81 is programmable to generate an electromagnetic wave at a predetermined frequency for a predetermined period of time according to user's requirement.

In some embodiments, a controller is provided to control the frequency and timing of electromagnetic waves.

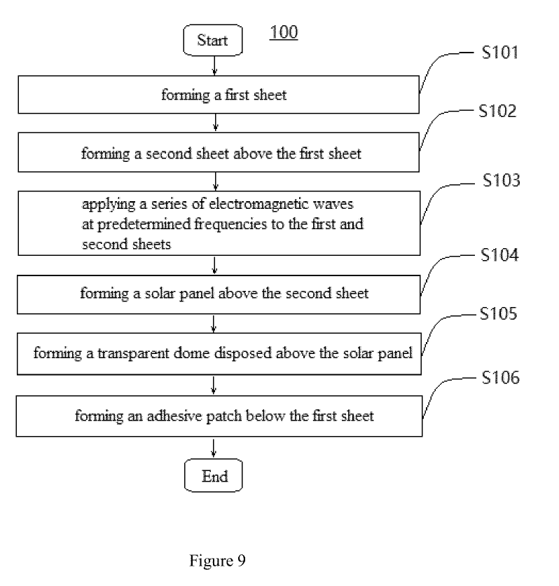

Referring to FIG. 9, in which a flow chart of a method 100 for manufacturing the patch is shown.

In step S101, a first layer is formed by using a material that is capable of storing electromagnetic energy. In some embodiments, the material for forming the first layer is crystalline carbon material. In some embodiments, the crystalline carbon material is blended with PET, wherein the PET is produced by polymerization of ethylene glycol and terephthalic acid. In step S102, a second layer is formed on the first layer by using a material that is capable of storing electromagnetic energy. In some embodiments, the material for forming the second layer is crystalline carbon material. In some embodiments, the first and second layers are formed as a whole. In some embodiments where there is only one of the first and second layers, step S101 or S102 may be omitted. In step S103, a series of electromagnetic waves at predetermined frequencies are applied to the first and/or second layers under a vacuum or partial vacuum condition. In some embodiments, the electromagnetic waves are generated by electromagnetic wave generator 81 (FIG. 7) towards the first layer and second layer placed in container 82 (FIG. 7). In step S104, a solar panel is formed above the second layer. In step S105, a transparent dome is formed above the solar panel. In step S106, an adhesive layer is formed below the first layer.

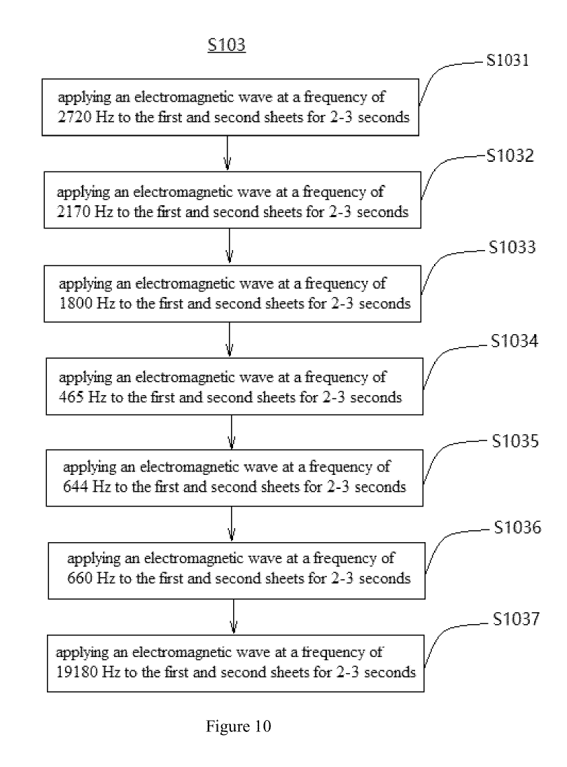

Referring to FIG. 10, in which a flowchart of a process of applying electromagnetic waves is shown. In step S1031, an electromagnetic wave at a frequency of 2720 Hz is applied to the first and second layers for about 2-3 seconds. In step S1032, an electromagnetic wave at a frequency of 2170 Hz is applied to the first and second layers for about 2-3 seconds. In step S1033, an electromagnetic wave at a frequency of 1800 Hz is applied to the first and second layers for about 2-3 seconds. In step S1034, an electromagnetic wave at a frequency of 465 Hz is applied to the first and second layers for about 2-3 seconds. In step S1035, an electromagnetic wave at a frequency of 644 Hz is applied to the first and second layers for about 2-3 seconds. In step S1036, an electromagnetic wave at a frequency of 660 Hz is applied to the first and second layers for about 2-3 seconds. In step S1037, an electromagnetic wave at a frequency of 19180 Hz is applied to the first and second layers for about 2-3 seconds. In some embodiments, one or more of above steps are omitted. In some embodiments, above steps are executed simultaneously, sequentially, or continuously.

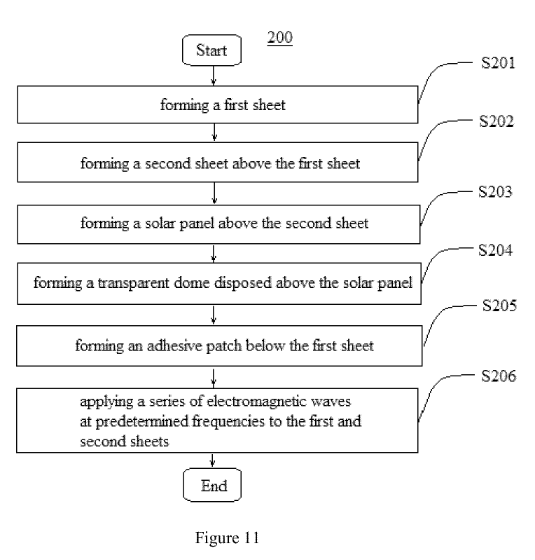

Referring to FIG. 11, in which a flow chart of a method 200 for manufacturing the patch is shown.

In step S201, a first layer is formed by using a material that is capable of storing electromagnetic energy. In some embodiments, the material for forming the first layer is crystalline carbon material. In some embodiments, the crystalline carbon material is blended with PET, wherein the PET is produced by polymerization of ethylene glycol and terephthalic acid. In step S202, a second layer is formed on the first layer by using a material that is capable of storing electromagnetic energy. In some embodiments, the material for forming the second layer is crystalline carbon material. In some embodiments, the first and second layers are formed as a whole. In some embodiments where there is only one of the first and second layers, step S201 or S202 may be omitted. In step S203, a solar panel is formed above the second layer. In step S204, a transparent dome is formed above the solar panel. In step S205, an adhesive layer is formed below the first layer. In step S206, a series of electromagnetic waves at predetermined frequencies are applied to the first and/or second layers under a vacuum or partial vacuum condition. In some embodiments, the electromagnetic waves are generated by electromagnetic wave generator 81 (FIG. 7) towards the first layer and second layer placed in container 82 (FIG. 7).

Since the first and/or second layers are made of a material capable of holding and releasing electromagnetic energy, the patch is able to hold electromagnetic energy after being applied with electromagnetic waves at different frequencies and slowly release the energy for different applications.

Depending on different applications such as EMR shielding, healthcare, combustion promotion, and so on, the frequencies, duration, and timing for applying those electromagnetic waves can be varied. For example, a patch releasing electromagnetic waves at a certain frequency may be used to for conditions related to the human body.

The final product, i.e., the patch, enhances the fuel to burn hotter by emitting electromagnetic energy with predetermined frequency towards the fuel. This process is called “frequency treatment”. With the increased burning at the flashpoint, less fuel is required to obtain torque and horsepower, which logically means fewer emissions from the vehicle's exhaust. Over time the consumer will also notice an increase in mileage due to increased combustion efficiency and temperature and the reduction of carbon deposits throughout the engine.

Effect of Patch on Combustion and Emission Characteristics of Hydrocarbon Fuels Purdue University investigated the effects of frequency treatment (i.e., electromagnetic energy treatment) of the patch of the present invention on combustion and emission characteristics of hydrocarbon fuels. In their experiment, a laminar co-flow ethylene (C₂H₄) flame was used as the surrogate of more complicated combustion process in Diesel (—CH₂—) engines. Ethylene (C₂H₄) is the simplest (—CH₂—) fuel, which produces soot when burning in the air due to combustion incompleteness. The complexities involved in Diesel engines, such as spray, evaporation, turbulent mixing, and multi-fuel components combustion, were eliminated by using a laminar flame. In a laminar co-flow ethylene flame, the soot emission is determined by the mixing of the air and fuel and by the combustion chemistry. Laser absorption measurement was used to obtain the soot volume fraction (fᵥ) distributions in the flame were estimated using the inverse interpretation of the transmittance measurements. The ethylene fuel was treated using the patch attached on the fuel storage cylinder.

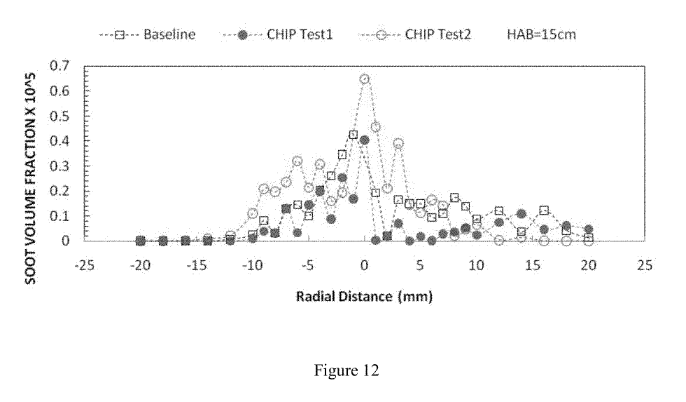

FIG. 12 depicts the soot volume fraction distributions in the flame at HAB (Heights Above the Burner) 15 cm for the “baseline” (without frequency treatment) and two “chip” (with frequency treatment using the “patch”) test campaigns, represented as baseline, CHIP Test 1, CHIP Test 2, respectively.

Soot volume fraction (soot concentration) distributions of the flames were estimated using the transmittance data to provide a straightforward interpretation of the effects of frequency treatment. Referring to FIG. 10, compared with the “baseline”, significantly decreased soot emission was observed in “CHIP Test 1”. The soot emission was increased in “CHIP Test 2”. This may be due to the duration (how long) the patch was applied. Since “CHIP Test 2” was conducted after the fuel was treated ten days longer than “CHIP Test 1”, the electromagnetic energy left in the patch may be less than that when it was ten days before.

This study indicates that frequency treatment affects the Diesel engine combustion process through both fuel/air mixing (physics) and fuel combustion chemistry.

Especially, when the “patch” was used, positive effect (reducing emission) of frequency treatment on soot emission or combustion completeness of the ethylene (C₂H₄) flame was observed.

What is claimed:

1. A patch emitting electromagnetic energy, comprising:
a first layer made of a first material, wherein the first material stores electromagnetic energy;
a second layer made of a second material, wherein the second material stores electromagnetic energy; and
the second layer is disposed above the first layer.

2. The patch of claim 1, further comprising:
a third layer made of a third material, wherein the third material harvests electromagnetic energy, wherein the third layer is disposed above the second layer.

3. The patch of claim 2, further comprising:
an adhesive layer disposed below the first layer.

4. The patch of claim 1, wherein the first and second materials comprise a crystalline carbon, wherein the crystalline carbon is at least one selected from the group consisting of graphite, graphene, and fullerene.

5. The patch of claim 4, wherein the crystalline carbon is fullerene.

6. The patch of claim 2, wherein the third material is a solar panel for harvesting electromagnetic energy in light.

7. The patch of claim 6, wherein a transparent dome is disposed above the solar panel.

8. The patch of claim 7, wherein a thickness of the first layer is about 0.5 mil, a thickness of the second layer is about 0.5 mil, and a thickness of the solar panel is about 1 mil.

9. The patch of claim 8, wherein a thickness of the transparent dome is about 1/16 inches.

10. The patch of claim 4, wherein the first and second materials are a blend of fullerene and a PET.

11. A method for manufacturing the patch of claim 1, comprising:
forming a first layer, wherein the first layer stores electromagnetic energy;
forming, above the first layer, a second layer, the second layer stores electromagnetic energy; and
applying a series of electromagnetic waves at predetermined frequencies to the first and second layers.

12. The method of claim 11, wherein the electromagnetic waves at the predetermined frequencies are applied under a vacuum condition.

13. The method of claim 12, further comprises:
forming a third layer, wherein the third layer harvests electromagnetic energy above the second layer.

14. The method of claim 13, further comprises:
forming a transparent dome above the third layer.

15. The method of claim 12, further comprises:
forming an adhesive layer below the first layer.

16. The method of claim 12, wherein the first layer is made of a blend of a crystalline carbon material and a PET, wherein the PET is produced by polymerization of ethylene glycol and terephthalic acid.

17. The method of claim 16, wherein the second layer is made of the blend of the crystalline carbon material and the PET.

18. The method of claim 12, wherein the step of applying the series of electromagnetic waves at the predetermined frequencies to the first and second layers under the vacuum condition comprises:
applying an electromagnetic wave at a frequency of 2720 Hz to the first and second layers for about 2-3 seconds;
applying an electromagnetic wave at a frequency of 2170 Hz to the first and second layers for about 2-3 seconds;
applying an electromagnetic wave at a frequency of 1800 Hz to the first and second layers for about 2-3 seconds;
applying an electromagnetic wave at a frequency of 465 Hz to the first and second layers for about 2-3 seconds;
applying an electromagnetic wave at a frequency of 644 Hz to the first and second layers for about 2-3 seconds;
applying an electromagnetic wave at a frequency of 660 Hz to the first and second layers for about 2-3 seconds; and
applying an electromagnetic wave at a frequency of 19180 Hz to the first and second layers for about 2-3 seconds.

19. The method of claim 16, wherein the crystalline carbon material is radio frequency sensitive.

20. The method of claim 13, wherein the third layer is a solar panel.
